(19) United States
(12) Patent Application Publication
(10) Pub. No.: US 2026/0153437 A1
(43) Pub. Date: Jun. 4, 2026

(54) MULTISPECTRAL PLASMONIC SENSOR ARRAY FOR CHIRAL MOLECULE DETECTION

(71) Applicant: University of Central Florida Research Foundation, Inc., Orlando, FL (US)

(72) Inventors: Debashis Chanda, Orlando, FL (US); Aritra Biswas, Orlando, FL (US)

(21) Appl. No.: 19/348,250

(22) Filed: Oct. 2, 2025

Related U.S. Application Data
(60) Provisional application No. 63/726,709, filed on Dec. 2, 2024.

(51) Int. Cl. G01N 21/31; G01N 21/19; G01N 21/25; G01N 21/35; G01N 33/52

(52) U.S. Cl. CPC G01N 21/3103; G01N 21/19; G01N 21/255; G01N 21/35; G01N 33/52; G01N 2021/258

(57) ABSTRACT

The invention relates to a multispectral plasmonic sensor for chiral molecular barcoding, employing a pixelated array of nanostructured plasmonic units coupled with a photonic cavity. Each pixel is tuned to resonate at distinct mid-infrared (mid-IR) wavelengths, facilitating simultaneous detection of molecular chiral and vibrational properties. The system generates near-perfect resonant absorption, maximizing electric and magnetic field enhancements. When illuminated with circularly polarized light, the sensor produces a chiral near-field with handedness determined by the polarization direction. The interaction between the sensor and chiral molecules is quantified using dissymmetry factors, forming unique chiral barcodes that encode molecular behavior across multiple wavelengths. The sensor enables surface-enhanced infrared absorption (SEIRA) induced surface-enhanced vibrational circular dichroism (SEVCD) measurements, providing comprehensive chiral and vibrational analysis. This approach creates a unique barcode for every chiral active molecule, supporting advanced applications in chemical analysis and molecular identification.

### CROSS-REFERENCE TO RELATED APPLICATIONS

This nonprovisional application claims the benefit of U.S. Provisional Application No. 63/726,709 entitled “MULTISPECTRAL PLASMONIC SENSOR ARRAY FOR CHIRAL MOLECULE DETECTION” filed Dec. 2, 2024, by the same inventors, all of which is incorporated herein by reference, in its entirety, for all purposes.

### GOVERNMENT INTEREST

This invention was made with Government support under Grant No. 1808045 awarded by the National Science Foundation. The Government has certain rights in the invention.

### BACKGROUND OF THE INVENTION

### 1. Field of the Invention

The described embodiments relate generally to plasmonic sensing technologies for molecular analysis. Specifically, the described embodiments relate to systems and methods for multispectral detection in the mid-infrared spectral range and molecular chiral barcoding to create unique identifier for chiral molecules.

### 2. Brief Description of the Prior Art

Chirality, the intrinsic property of asymmetry, is a fundamental aspect of our world, sparking renewed interest across various research disciplines, including chemistry, biology, physics, medicine, and pharmaceutics in recent decades. It describes the property of an object being non-overlapping with its mirror image. Almost half of all designed medical drugs along with a plethora of naturally occurring biomolecules are chiral, resulting in an ever-growing demand for efficient detection and classification techniques. Chirality can exist at the smallest molecular bonding level (tryptophan, glucose, limonene to name a few) or can exist at a large-scale structurally complex level such as in supramolecular conformations (proteins, DNAs, peptide chains, etc.). Certain pairs of chiral molecules, known as enantiomers, exist in both mirror imaged forms, and although they exhibit similar physical properties, they may have vastly different chemical or biological implications with regards to reactivity, potency, and functionality.

Chiroptical measurement techniques such as optical rotatory dispersion (ORD) and circular dichroism (CD) have been excellent candidates for identification of such complex chiral structures and molecules, where polarized light having an associated spin-momentum (such as in circularly polarized light) interacts differently with chiral molecules, enabling the identification and reliable determination of their chiral properties. However, reliable CD spectral measurement of low molecular concentrations, especially in the infrared domain, is difficult owing to weak extinction cross-sections due to dimensional mismatch between target molecules and excitation wavelength. Hence, higher concentration and larger volume (longer interaction path length) are required for reliable CD measurements.

Current methods and systems for detecting chiral light-matter interactions employ both plasmonic and photonic platforms to amplify optical response through enhanced chiral interaction. Chiral sensing platforms that require chiral nanostructures of varying degrees of asymmetry can be challenging to replicate. Though a significant focus has been put in enhancing CD responses in the ultra-violet (UV), visible, and near-infrared regions, very few have shown applications in the mid-infrared (mid-IR) domain, where almost all molecules exhibit unique vibrational absorption “fingerprints.” This region carries important information about chemical bonds as well as macromolecular arrangements of large molecules like proteins. Though there have been few attempts to develop techniques for the detection and identification of such molecular mid-IR fingerprints, one main limitation yet to overcome is the inability to distinguish spectrally similar molecules.

Many long-chain biomolecules have similar vibrational spectra in the mid-IR region due to them having similar amino-acid blocks but different three-dimensional block orientations and arrangements which impart spatial asymmetry, making them chiral active. Several large macromolecules have higher order secondary and tertiary structural forms. These structural arrangements cannot be distinguished by simple spectral absorption analysis, but are important as they can unveil information about disorders and misfolding in proteins, and accurate structural understanding may enable diagnosis of several neurodegenerative diseases like dementia, Parkinson's diseases, etc. Vibrational circular dichroism (VCD) identifies such structural arrangements uniquely by determining the asymmetry in molecular interaction with oppositely handed circularly polarized light (CPL). However, conventional bulk-phase VCD suffers from the large mismatch between the detection wavelength and the molecular dimension, leading to extremely weak differential signals typically of the order of 10⁻⁵-10⁻⁶, which enforces the need for high concentration and longer detection pathlengths to obtain a reliable signal.

Accordingly, what is needed is a detection scheme that would enable sensitive and unique identification of chiral arrangements from low concentration analytes in the mid-IR vibrational fingerprint domain. However, in view of the art considered as a whole at the time the present invention was made, it was not obvious to those of ordinary skill in the field of this invention how the shortcomings of the prior art could be overcome.

### SUMMARY OF THE INVENTION

The long-standing but heretofore unmet need for reliable detection and characterization of chiral active molecules is now met by a novel and non-obvious invention disclosed and claimed herein. In an aspect, the present disclosure pertains to a multispectral plasmonic sensor and associated system for generating a molecular barcode of a chiral active molecule. In an embodiment, the multispectral plasmonic sensor comprises: (a) a substrate; (b) a reflector layer disposed about a top surface of the substrate; (c) a dielectric cavity layer disposed about a surface of the reflector layer; (d) a pixelated array comprising a plurality of pixels disposed about a surface of the dielectric cavity layer, wherein each pixel comprises a periodic square array of nanoholes; (e) a plurality of metallic nano disks disposed above the nanoholes of the pixels to define a plurality of hole-disk plasmonic resonators; and/or (f) wherein the pixels of the pixelated array are scaled in increments across the array such that each pixel resonates at a distinct mid-infrared (mid-IR) wavelength.

In some embodiments, the pixelated array may comprise a 5 by 5 arrangement of pixels, with each pixel having lateral dimensions of approximately 1 mm by 1 mm. In some other embodiments, the nanoholes of the pixels may maintain a fixed diameter-to-periodicity ratio of 2:3, while being scaled in increments of about 5% in successive pixels to generate distinct resonances across the mid-IR region. Additionally, in some embodiments, the reflector layer may comprise a titanium adhesion layer and a gold layer, and the dielectric cavity layer may comprise silicon dioxide.

Another aspect of the present disclosure pertains to a system for generating a molecular barcode of a chiral active molecule. In an embodiment, the system comprises: (a) a multispectral plasmonic sensor according to any embodiment described herein; (b) a light source configured to generate mid-IR circularly polarized light directed towards the pixelated array; and/or (c) a detection system communicatively coupled to the pixelated array and configured to measure spectral responses of the pixelated array under left-circularity and right-circularly polarized illumination. In this manner, the detection system may also compute dissymmetry factors for the pixelated array and/or assemble the dissymmetry factors into a chiral barcode.

Moreover, in some embodiments, the detection system comprises (a) a reflection-mode microscope having a low numerical aperture; (b) a Fourier transform infrared spectrometer with a mid-IR thermal source and/or a potassium bromide beamsplitter; (c) a quarter-wave plate mechanically coupled to a motorized rotation stage; and/or (d) a cryo-cooled mercury-cadmium telluride detector.

Furthermore, in some embodiments, the system may further comprise a processor configured to compare the generated barcode to a library of stored chiral barcodes to classify the chiral active molecule. In some embodiments, the sensor may enable simultaneous surface-enhanced infrared absorption (“SEIRA”) and surface-enhanced vibrational circular dichroism (“SEVCD”) measurements thereby allowing comprehensive analysis of molecular vibrational and chiral properties.

In another aspect, the present disclosure pertains to a method for detecting and characterizing chiral active molecules. In some embodiments, the method comprises: (a) providing a system for generating a molecular barcode of a chiral active molecule, the system comprising a multispectral plasmonic sensor, a light source configured to generate mid-IR circularly polarized light, and a detection system configured to measure spectral responses of the sensor; (b) illuminating the multispectral plasmonic sensor with circularly polarized light to generate enhanced electric and magnetic near fields at each pixel of the pixelated array; (c) introducing a chiral active molecule onto the surface of the pixelated array; (d) measuring, via the detection system, the differential absorption of left- and right-circularly polarized light; (e) computing dissymmetry factors for each pixel of the pixelated array; and/or (f) assembling the dissymmetry factors into a barcode representing a molecularly unique chiral signature of the introduced chiral active molecule.

In some embodiments, the method may further comprise the step of depositing the chiral active molecule as a thin film on the surface of the pixelated array. Additionally, in some embodiments, the method may comprise the step of integrating dissymmetry values across spectral regions corresponding to molecular absorption bands and/or differentiating enantiomers of the same chiral molecule based on distinct barcode patterns. In some embodiments, the method may further comprise calibrating the multispectral plasmonic sensor prior to introduction of the analyte by measuring baseline spectral responses of the pixelated array under left-circularity and right-circularly polarized illumination in the absence of any analyte.

In some embodiments, the method may further comprise depositing the chiral active molecule as a thin film on the surface of the pixelated array, integrating dissymmetry values across spectral regions corresponding to molecular absorption bands, or differentiating enantiomers of the same chiral molecule based on distinct barcode patterns. In some embodiments, the method may further comprise calibrating the multispectral plasmonic sensor prior to introduction of the analyte by measuring baseline spectral responses of the pixelated array under left-circularity and right-circularly polarized illumination in the absence of any analyte.

An additional aspect of the present disclosure pertains to a method for fabricating a multispectral plasmonic sensor. In an embodiment, the method comprises: (a) providing a master pattern comprising a pixelated pattern of scaled nanohole arrays; (b) forming a poly(dimethylsiloxane) (“PDMS”) stamp mold from the master pattern; (c) preparing a substrate by cutting a glass slide, sonicating the glass slide in acetone, rinsing with isopropyl alcohol and deionized water, and/or drying with inert nitrogen; (d) depositing by electron-beam evaporation a first titanium adhesion layer, a gold layer, and a second titanium adhesion layer on the substrate; (e) depositing an amorphous silicon dioxide layer on the substrate by electron-beam evaporation; (f) spin-coating a photoresist onto the silicon dioxide layer and embossing the photoresist with the PDMS stamp mold to transfer the nanohole array pattern; (g) UV-curing the embossed photoresist; (h) etching the substrate in an argon/trifluoromethane (“Ar/CHF₃”) environment to transfer the nanohole pattern into the silicon dioxide cavity; and/or (i) depositing by electron-beam evaporation a metallic layer comprising titanium and gold to form a plurality of nano disks disposed above the nanoholes to define a plurality of hole-disk plasmonic resonators.

Additionally, in some embodiments, the master pattern design may include a 5 by 5 array of pixels such that each pixel may comprise a square array of nanoholes. Additionally, in some embodiments, square array of nanoholes may comprise a size about 1 mm by 1 mm.

Moreover, in some embodiments, the amorphous silicon dioxide may compromise layer of about 1 μm thickness on the substrate. Furthermore, in some embodiments, the nanohole pattern may be transferred into the silicon dioxide cavity to a depth of about 200 nm.

In some embodiments, the nanoholes of the smallest pixel have a diameter of about 0.464 μm and/or a periodicity of about 0.696 μm thereby corresponding to a diameter-to-periodicity ratio of about 2:3. In some embodiments, the successive pixels of the array are scaled in increments of about 5% in both nanohole size and periodicity to generate distinct mid-infrared resonances across the pixelated array.

Applications include distinguishing between enantiomers based on their spectral profiles and analyzing complex biomolecular structures. In this manner, the system and method may leverage advanced techniques in plasmonic sensing to enhance the sensitivity and specificity of molecular detection, supporting diverse fields such as pharmaceutics, chemistry, and biology.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not restrictive.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is a representation of a multispectral plasmonic sensor, according to an embodiment of the present disclosure.

FIG. 2 is a photographic image of a fabricated multispectral plasmonic sensor, magnified to detail a pixelated surface with each pixel of the pixelated surface, according to an embodiment of the present disclosure.

FIG. 3 is a representation of a unit cell of the multispectral plasmonic sensor, according to an embodiment of the present disclosure.

FIG. 4 is a representation of a pixel configuration of the multispectral plasmonic sensor and corresponding scanning electron microscope images of said pixel 1 and pixel 25, labelled accordingly as well as a cross-section scanning electron microscope (“SEM”) image of the geometry of the pixel configuration, according to an embodiment of the present disclosure.

FIG. 5 is a graphical illustration depicting a spectral reflectance and a corresponding dissymmetry map of the pixelated sensor without any molecule, according to an embodiment of the present disclosure.

FIG. 6 is a graphical illustration depicting a spectral reflectance and corresponding dissymmetry map of the pixelated sensor without adsorbed molecules and with the molecular vibrational absorption fingerprint, according to an embodiment of the present disclosure.

FIG. 7 is a graphical illustration depicting a visualization of a 3D near-field optical chiral density over a unit cell of the multispectral plasmonic sensor for left- and right-circularly polarized light (“CPL”), according to an embodiment of the present disclosure.

FIG. 8A is graphical illustration depicting a 2D profile of an electric (|E/E₀|²) and a magnetic (|H/H₀|²) field intensity enhancement first at 3 nm above a hole for one of the pixels of the pixelated array of the plasmonic sensor at the first-order localized surface plasmon resonance (“LSPR”), according to an embodiment of the present disclosure.

FIG. 8B is a graphical illustration depicting a 2D profile of a an electric (|E/E₀|²) and a magnetic (|H/H₀|²) field intensity enhancement at a cross-section through the center for one of the pixels of the pixelated array of the plasmonic sensor at the first-order localized surface plasmon resonance (“LSPR”), according to an embodiment of the present disclosure.

FIG. 9 is graphical plot depicting a volume integrated optical chirality up to a lateral height of 200 nm above the surface for a pixelated plasmonic system, according to an embodiment of the present disclosure.

FIG. 10 is a graphical plot depicting a corresponding LSP resonance wavelength and full width at half maximum (“FWHM”), according to an embodiment of the present disclosure.

FIG. 11A is a graphical plot depicting a system far-field response before and after molecular adsorption for four pixels of the plasmonic sensor with various degrees of overlaps (S=2, 13, 21, 25), according to an embodiment of the present disclosure.

FIG. 11B is a graphical plot depicting a corresponding dissymmetry for four pixels as displayed in FIG. 11A, according to an embodiment of the present disclosure.

FIG. 11C is a graphical plot depicting a corresponding barcode map of the four pixels as displayed in FIGS. 1A-11B, according to an embodiment of the present disclosure.

FIG. 11D is a graphical depiction of the location of the four pixels selected as displayed in FIGS. 11A-11C, according to an embodiment of the present disclosure.

FIG. 12A is the chiral barcoding-based discrimination of enantiomers by an infrared reflection-absorption spectroscopy (“IRRAS”) measurement of adsorbed thin-film thalidomide enantiomers (R—(top) and S—(bottom)) on gold coated substrates, according to an embodiment of the present disclosure.

FIG. 12B depicts corresponding images of reflectance heat-maps for R- and S-thalidomide-adsorbed pixelated metasurfaces for RCP illumination, according to an embodiment of the present disclosure.

FIG. 13 is a graphical illustration depicting a color barcoding of integrated dissymmetry for thalidomide enantiomers, R—(top) and S—(bottom) with pixel-to-pixel distance for the barcodes and showing dissymmetry distances for each pixel, according to an embodiment of the present disclosure.

FIG. 14A is a graphical illustration depicting a chiral barcoding-based classification of spectroscopically similar biomolecules by IRRAS measurement of hemoglobin (top) and lysozyme (bottom) on gold coated substrates, according to an embodiment of the present disclosure. with corresponding heatmaps and color barcoding, according to an embodiment of the present disclosure.

FIG. 14B depicts corresponding heatmaps of the chiral barcoding-based classification of spectroscopically similar biomolecules by IRRAS measurement of hemoglobin (top) and lysozyme (bottom) on gold coated substrates as shown in FIG. 14A, according to an embodiment of the present disclosure.

FIG. 14C depicts corresponding color barcoding of integrated dissymmetry of the chiral barcoding-based classification of spectroscopically similar biomolecules by IRRAS measurement of hemoglobin (top) and lysozyme (bottom) on gold coated substrates as shown in FIGS. 14A-14B, according to an embodiment of the present disclosure.

FIG. 15 is a graphical illustration depicting a detection system for spectral dissymmetry measurement, according to an embodiment of the present disclosure.

FIG. 16 is a flow chart depicting the steps of a method detecting and characterizing chiral active molecules, according to an embodiment of the present disclosure.

FIG. 17 is a flow chart depicting the steps of a method of fabricating a multispectral plasmonic sensor for detecting and characterizing chiral molecules, according to an embodiment of the present disclosure.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention.

As such, elements/components shown in diagrams are illustrative of exemplary embodiments of the disclosure and are meant to avoid obscuring the disclosure. Any headings, used herein, are for organizational purposes only and shall not be used to limit the scope of the description or the claims.

Furthermore, the use of certain terms in various places in the specification, described herein, are for illustration and should not be construed as limiting. For example, any reference to an element herein using a designation such as “first,” “second,” and so forth does not limit the quantity or order of those elements, unless such limitation is explicitly stated. Rather, these designations may be used herein as a convenient method of distinguishing between two or more elements or instances of an element. Therefore, a reference to first and/or second elements does not mean that only two elements may be employed there or that the first element must precede the second element in some manner. Also, unless stated otherwise a set of elements may comprise one or more elements.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

Referring in general to the following description and accompanying drawings, various embodiments of the present disclosure are illustrated to show its structure and method of operation. Common elements of the illustrated embodiments may be designated with similar reference numerals.

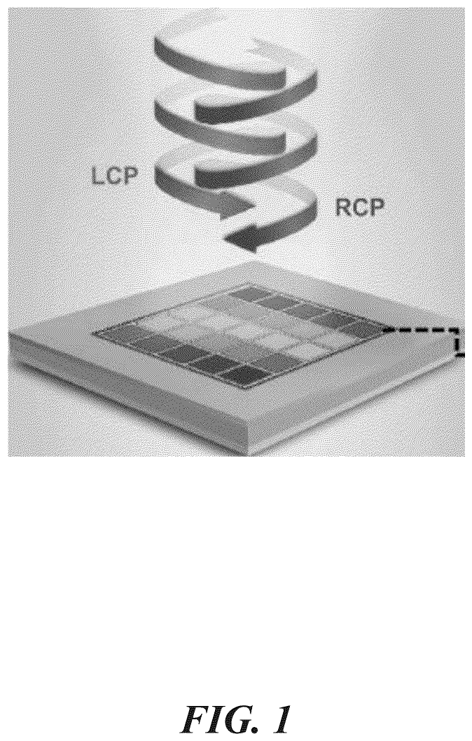

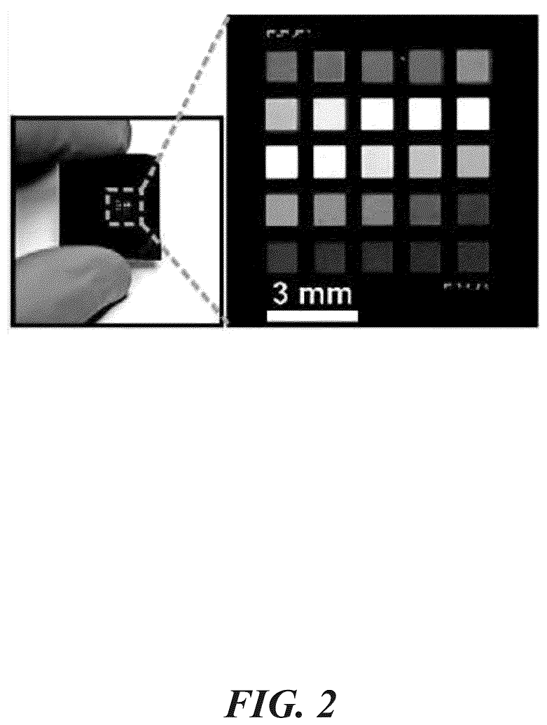

Accordingly, the relevant descriptions of such features apply equally to the features and related components among all the drawings. For example, any suitable combination of the features, and variations of the same, described with components illustrated in FIG. 1, can be employed with the components of FIG. 2, and vice versa. This pattern of disclosure applies equally to further embodiments depicted in subsequent figures and described hereinafter. It should be understood that the figures presented are not meant to be illustrative of actual views of any particular portion of the actual structure or method but are merely idealized representations employed to more clearly and fully depict the present invention defined by the claims below.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

In the following description, for the purposes of explanation, numerous specific details are set forth in order to provide a thorough understanding of embodiments of the present technology. It will be apparent, however, to one skilled in the art that embodiments of the present technology may be practiced without some of these specific details.

The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compacts disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

### Glossary of Claim Terms

Barcode refers to a unique spectral fingerprint created from the integrated dissymmetry factors of a chiral molecule, encoding its behavior across multiple infrared wavelengths. The barcode allows for precise identification and differentiation of chiral molecules by capturing their distinct vibrational and chiral responses using the multispectral plasmonic sensor. This method provides a comprehensive molecular profile, enabling high-sensitivity analysis and facilitating the detection of complex chiral mixtures or biomolecular structures.

Chiral Active Molecule refers to a molecule exhibiting optical activity, where it interacts differently with left- and right-circularly polarized light, causing variations in absorption or reflection. The system detects and analyzes this activity using plasmonic enhancement techniques, allowing for high-sensitivity characterization of the molecule's chirality, making it ideal for applications requiring enantiomeric discrimination and detailed molecular characterization.

Chiral Near-Field refers to the enhanced electromagnetic field generated around the plasmonic nanostructures when illuminated by circularly polarized light. This near-field exhibits single-handed chirality, aligning with the handedness of the incident light, and amplifies the interaction with chiral molecules to improve detection sensitivity.

Circular Dichroism (“CD”) refers to the differential absorption of left- and right-circularly polarized light by chiral molecules. This property is used to quantify the chiral behavior of a molecule by measuring how much more one circular polarization (either left or right) is absorbed compared to the other. The CD signal is further enhanced using surface-enhanced vibrational circular dichroism (“SEVCD”) to detect and analyze chiral molecules with greater sensitivity.

Circularly Polarized Light (“CPL”) refers to light in which the electric field vector rotates in a circular pattern, either clockwise (i.e., right-handed “RCP”) or counterclockwise (i.e., left-handed “LCP”). This configuration efficiently interacts with the plasmonic nanostructures to enhance the detection and analysis of chiral molecules, allowing for differential absorption measurements that reveal the molecule's chiral characteristics.

Far-Field Near-Perfect Resonant Absorption refers to a condition where nearly all incident light energy is absorbed at specific mid-IR resonance frequencies, facilitated by the hybrid coupling of plasmonic nanostructures and a photonic cavity. This effect maximizes electric and magnetic field enhancements, enhancing the interaction between the sensor and target molecules and improving signal strength for spectroscopic analysis.

Localized Surface Plasmon Resonance (“LSPR”) refers to the collective resonant oscillation of conduction electrons in a plasmonic nanostructure, excited by incident light at specific wavelengths. LSPR generates highly localized and enhanced electromagnetic fields at the nanostructure's surface, significantly improving the interaction between light and chiral molecules. This enhancement facilitates the detection and analysis of weak chiral and vibrational signals, making the sensor highly sensitive to molecular characteristics.

Nanostructured Pixels refers to individual sensing units arranged in a pixelated array, each tuned to resonate at a specific mid-IR wavelength. These pixels work collectively to capture the unique chiral and vibrational responses of molecules, enabling simultaneous multispectral detection. The array generates a chiral barcode that encodes the molecular behavior across multiple wavelengths, providing a comprehensive analysis of chiral molecules or mixtures.

Optical Chiral Density (“C”) refers to a measure of the chirality of an electromagnetic field, defined by the degree of “twist” in the field's structure. In the described system, optical chiral density is generated and enhanced at resonance, creating a homogeneous, single-handed near-field. The direction of this chiral density depends on the handedness of the incident circularly polarized light, which enhances the sensor's ability to detect chiral molecules.

Photonic Cavity refers to a structure integrated with the plasmonic system to confine and enhance electromagnetic fields at resonance frequencies. The photonic cavity ensures sharp and narrow resonance peaks, amplifying both electric and magnetic fields. This enhancement maximizes light-matter interactions for detecting and analyzing the vibrational and chiral characteristics of molecules with high sensitivity and efficiency.

Surface-Enhanced Infrared Absorption (“SEIRA”) refers to a technique that uses plasmonic nanostructures to significantly amplify the infrared absorption signals of molecules. SEIRA enhances the sensitivity of vibrational spectroscopic measurements, making it possible to detect and analyze even weak absorption features. In the described sensor, SEIRA is used alongside SEVCD for comprehensive molecular analysis.

Surface-Enhanced Vibrational Circular Dichroism (“SEVCD”) refers to a method that amplifies the vibrational circular dichroism signals of chiral molecules using plasmonic nanostructures. SEVCD is performed simultaneously with SEIRA, providing a robust technique for analyzing chiral and vibrational properties. This combination allows for detailed, high-sensitivity characterization of chiral molecules in the mid-IR spectrum.

As used herein, the terms “about,” “approximately,” or “roughly” refer to being within an acceptable error range (i.e., tolerance) for the particular value as determined by one of ordinary skill in the art, which will depend in part on how the value is measured or determined (e.g., the limitations of a measurement system) (e.g., the degree of precision required for a particular purpose, such as packaging and/or delivery of at least one prosthesis and/or prosthetic implant into a surgical pocket). As used herein, “about,” “approximately,” or “roughly” refer to within +25% of the numerical.

All numerical designations, including ranges, are approximations which are varied up or down by increments of 1.0, 0.1, 0.01 or 0.001 as appropriate. It is to be understood, even if it is not always explicitly stated, that all numerical designations are preceded by the term “about.” It is also to be understood, even if it is not always explicitly stated, that the compounds and structures described herein are merely exemplary and that equivalents of such are known in the art and can be substituted for the compounds and structures explicitly stated herein.

Wherever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “no more than,” “less than,” or “less than or equal to” precedes the first numerical value in a series of two or more numerical values, the term “no more than,” “less than” or “less than or equal to” applies to each of the numerical values in that series of numerical values. For example, less than or equal to 1, 2, or 3 is equivalent to less than or equal to 1, less than or equal to 2, or less than or equal to 3.

### Multispectral Plasmonic Sensor for Chiral Molecules

The present disclosure pertains to a multispectral plasmonic sensor (“sensor”) and associated systems and/or related methods for detecting and fabricating the sensors. Accordingly, the sensor can detect chiral active molecules using pixel-resolved resonant responses in the mid-infrared (“mid-IR”) spectral region. In some embodiments, the sensor can comprise a substrate having a reflector layer on at least one surface of the substrate. Moreover, the sensor can include a dielectric cavity layer disposed on a surface of the reflector layer such that an array of pixels may be mechanically coupled onto a surface of the dielectric cavity layer.

As shown in FIG. 1, in some embodiments, the multispectral plasmonic sensor comprises a substrate. Accordingly, the substrate may comprise a glass slide. In some other embodiments, the substrate may comprise semiconductors such as silicon and/or gallium arsenide, polymers such as polyethylene terephthalate (“PET”), flexible substrates such as polyimide, and/or a combination thereof. In this manner, the user may select a substrate depending on the qualities and/or active properties of the chiral active molecule being analyzed.

Moreover, in some embodiments the substrate can include an adhesion layer disposed about a top surface of the substrate. In some embodiments, the adhesion layer disposed about a top surface of the substrate may comprise of titanium and/or a titanium alloy. Additionally, a reflector layer may be disposed about the surface of the adhesion layer and/or substrate. Accordingly, in some embodiments, the reflector layer may comprise of gold and/or a gold alloy.

Moreover, in some embodiments, the sensor includes a dielectric cavity layer disposed about a top surface of the reflector layer. Accordingly, the dielectric cavity layer may comprise silicon dioxide (SiO₂) and/or other dielectric materials known in the art, including but not limited to aluminum oxide, hafnium oxide, or silicon nitride. In this manner, these dielectric materials may be used in alternative embodiments to tailor the refractive index or thermal stability depending on the desires of the user and/or chiral active material being analyzed.

Additionally, in some embodiments, the thickness of the dielectric cavity layer may be selected to enhance optical confinement and improve coupling efficiency between incident radiation and localized surface plasmon resonances generated in the pixelated array. In this manner, the dielectric cavity may have a thickness ranging from about 10 nanometers to about 300 nanometers. For example, in some embodiments, the thickness of about 160 nanometers, however this example should not be interpreted as limiting as to other potential thicknesses.

Accordingly, a thinner dielectric layer may provide stronger near-field confinement and/or sharper resonance peaks whereas a thicker dielectric layer may support broader spectral tunability. Moreover, the dielectric thickness may be varied across different regions of the sensor to optimize performance for different classes of molecules and/or regions of the mid-IR spectrum.

As best depicted in FIG. 1, in some embodiments, the sensor can include a pixelated array disposed above the dielectric cavity layer such that the pixelated array comprises a plurality of pixels. In this manner, the pixels of the pixelated array may comprise a periodic square array of nanoholes having metallic nano disks disposed above each nanohole.

As such, the nanoholes and/or metallic nano disks define one or more hole-disk plasmonic resonators configured to produce resonant features when illuminated with mid-IR light. Moreover, in some embodiments, the nanoholes may have a diameter-to-periodicity ratio of about 2:3 across the pixelated array although alternative ratios may be utilized by a user depending on desired resonance strength of specific chiral active molecules (i.e., “analyte”) being analyzed.

In some embodiments, the pixelated array can be configured as a 2 by 2 matrix to about a 100 by 100 matrix. For example, in some embodiments, the pixelated array is configured as a 5 by 5 matrix thereby resulting in twenty-five distinct pixels in the pixelated array. In some embodiments, the pixels of the pixelated array may cover an area of about 1 mm by 1 mm. Moreover, the pixels of the pixelated array may cover large and/or smaller areas depending on the aspects of the chiral active molecule being analyzed. In this manner the pixels may be used to increase spectral coverage, resolution, and/or a combination thereof.

Moreover, in some embodiments, the pixels of the pixelated array can be scaled incrementally in size. In this manner, the nanohole diameter and/or periodicity of each pixel may be increased by about 5% between each successive pixels, enabling each pixel to resonate at a distinct mid-IR wavelength. In some other embodiments, the pixels may not be scaled and/or scaled at a greater successive rate depending on the desires of the user and properties of the chiral active molecule being analyzed.

In this manner, the scaling of the pixelated array as previously described, may allow for coverage of the vibrational “fingerprint” region of the mid-IR spectrum. Accordingly, the vibrational region provides high specificity for molecular identification due to characteristic vibrational absorption bands. Moreover, in some other embodiments, various other scaling increments may be utilized such as non-linear percentage changes and/or adaptive scaling algorithms that may be used to concentrate spectral coverage in regions of particular analytical interest for a user. In this manner, these different approaches approach allows the pixelated array to be tailored for specific classes of molecules while still maintaining general-purpose capability.

As shown in FIG. 1, in some embodiments, the sensor is configured to interact with circularly polarized light. The illumination of the array with left-circularly polarized (“LCP”) and right-circularly polarized (“RCP”) mid-IR light can produce distinct local electromagnetic field distributions at the surface of the pixels of the pixelated array. Accordingly, the adsorption of a chiral active molecule produces differential dissymmetry factors across the pixel array enabling for enantiomer-specific detection.

In some other embodiments, the sensor can include a protective overlayer, such as a transparent polymer and/or an oxide layer configured to provide a shield for the nanostructures while still permitting analyte adsorption. In this manner, the protective overlayer may prevent fouling of the sensor, extend device longevity, enable repeated sensor use, and/or a combination thereof. In some other embodiments, functionalized surface chemistries including but not limited to self-assembled monolayers and/or molecular binding ligands can be applied to the top surface of the pixels of the pixelated array to enhance selectivity toward specific classes of chiral active molecules such as biomolecules, pharmaceuticals, and/or agrochemicals.

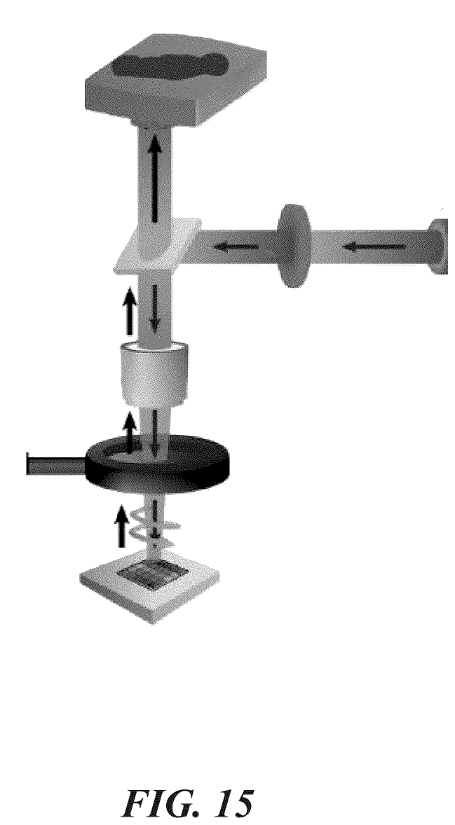

As depicted in FIG. 15, in conjunction with FIG. 1, the present disclosure includes a system for generating molecular barcodes (i.e., “system”) comprising the multispectral plasmonic sensor described herein above. Moreover, the system may further comprise a light source and/or a detection subsystem (i.e., “detection system”).

In some embodiments, the light source may comprise a broadband mid-IR thermal emitter. In some other embodiments, the light source may comprise a cascade laser and/or a tunable optical parametric oscillator as alternative and/or supplemental light sources to enhance spectral resolution and/or intensity. Moreover, in some embodiments, the polarization of the light source may be controlled by a quarter-wave plate mounted on a motorized stage. Accordingly, the quarter-wave plate and the motorized stage allow for the production and/or alternating of the system between LCP and RCP light.

As shown in FIG. 15, in some embodiments, the detection system can include a reflection-mode microscope having a low numerical aperture objective positioned in a manner to collect reflected light from the sensor. Accordingly, the reflection-mode microscope may be coupled to a Fourier transform infrared spectrometer configured with a potassium bromide beamsplitter.

Additionally, in some embodiments, the detection system comprises a telluride detector configured to acquire the reflected signals across the mid-IR band. In some embodiments the telluride detector may comprise a cryo-cooled mercury-cadmium telluride (“MCT”) detector. In some alternative embodiments, room-temperature detectors such as pyroelectric and/or bolometric sensors may be used to provide cheaper and/or portable configurations of the detection system.

In some embodiments, the detection system is configured to measure a pixel-resolved reflectance spectra under alternating LCP and RCP illumination. In some embodiments, a processor can be communicatively coupled to the detection system such that the processor may calculate and/or determine dissymmetry factors for each pixel of the pixelated array using reflectance intensity values gathered from the paired polarization states. In this manner, these dissymmetry values are then compiled across the pixel array to generate a digital representation in the form of a molecular barcode. Accordingly, the barcode captures the distinct chiral signature of the molecule adsorbed on the sensor surface.

In some embodiments, the MCT detector may be configured to obtain and/or measure the pixel-resolved reflectance spectra under alternating LCP and RCP illumination and generate a digital representation of the dissymmetry values in the form of a molecular barcode. In some other embodiments, the collected data may be transferred from the MCT detector and/or processor to an external computing device which is able to perform the appropriate calculations regarding the dissymmetry values to generate the molecular barcode of the chiral active molecule being analyzed.

In some embodiments, the processor can compare the generated molecular barcode to a stored library of reference barcodes to classify the studied chiral active molecule. In this manner, the system may recognize molecular enantiomers and/or closely related molecules with overlapping mid-IR absorption spectra. Moreover, machine learning algorithms may be integrated to improve classification accuracy and enable adaptive expansion of the library as new barcodes are acquired.

Additionally, in some other embodiments, the molecular barcode may represent an integration of dissymmetry values across the pixelated array. In some embodiments, the barcode may be visualized on an external computing device for a user as a two-dimensional grid with grayscale and/or color-coded elements corresponding to dissymmetry magnitude at each pixel. In some other embodiments, the barcode may be stored as a numerical data file for digital processing and/or classification.

Furthermore, in some embodiments, the system may further comprise a fluidic delivery mechanism, including but not limited to one or more microfluidic channels and/or spray deposition systems configured to introduce the chiral active molecules onto the plasmonic sensor surface in a controlled and consistent manner. As such, the fluidic delivery mechanism may enable integration of the sensor into automated workflows for sample preparation and/or continuous monitoring.

In some other embodiments, the metallic layers of the reflector and/or nano disks may comprise other conductive materials. In this manner the reflector layer may further comprise of conductive materials including but not limited to silver, aluminum, platinum, multilayer alloys, and/or a combination thereof. Moreover, the nano disks may also be comprised of transparent conductive oxides which may extend the spectral tunability and/or enable electrical biasing of the sensor.

### Method for Detecting and Characterizing Chiral Active Molecules

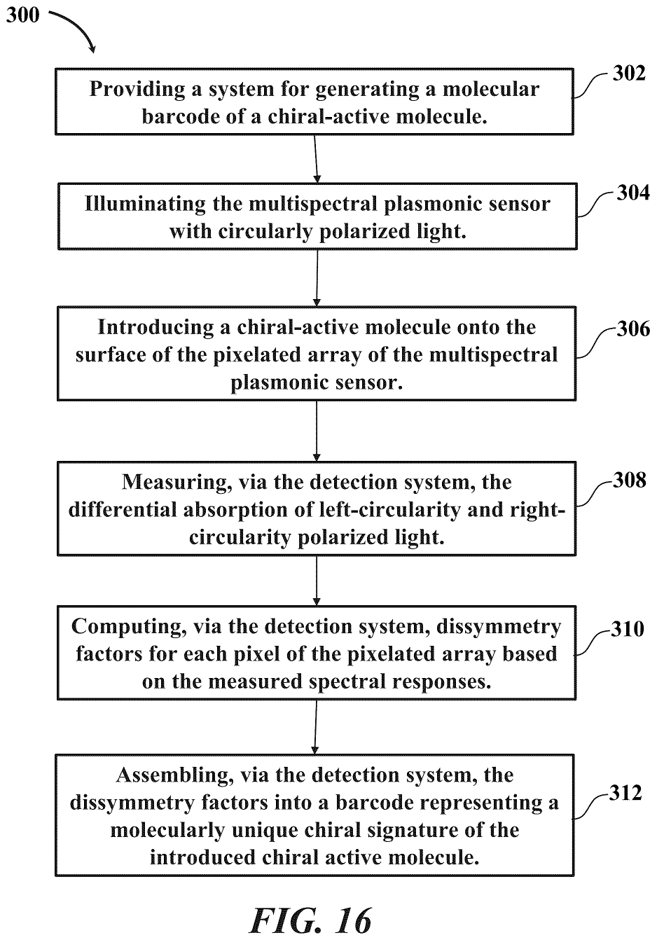

Referring now to FIG. 16, in conjunction with FIGS. 1 and 15, a method 300 is depicted for detecting and characterizing chiral active molecules using the multispectral plasmonic sensor and/or the detection system. The steps delineated in method 300 are merely exemplary of a preferred order of operation and may be performed in alternative orders, with additional steps, or with steps omitted depending on the embodiment of the sensor and/or detection system utilized.

As shown in FIG. 16, in conjunction with FIG. 1 and FIG. 15, method 300 begins with step 302, providing a system for generating a molecular barcode of a chiral active molecule (“system”). In some embodiments, the system can comprise a multispectral plasmonic sensor including a substrate. In this manner, the system may include a reflector layer disposed about a top surface of the substrate such that a dielectric cavity layer can be disposed about a surface of the reflector layer.

Moreover, a pixelated array comprising a plurality of pixels may be disposed about a surface of the dielectric cavity layer such that each pixel may comprise a periodic square array of nanoholes and/or a plurality of metallic nano disks disposed above one or more nanoholes thereby defining one or more hole-disk plasmonic resonators scaled in increments across the pixelated array. In some embodiments, the system includes a light source configured to generate mid-infrared circularly polarized light and/or a detection system communicatively coupled to the pixelated array configured to measure pixel-resolved spectral responses under left- and right-circularly polarized illumination.

The next step, step 304, in some embodiments, method 300 includes illuminating the multispectral plasmonic sensor with a circularly polarized light. In some embodiments, a processor may be operably coupled to the detection system such that the processor can command rotation stage of the detection system. In this manner, the processor may set a quarter-wave plate of the detection system for left-handed circular polarization and/or right-handed circular polarization in a predetermined sequence while a microscope objective directs the illumination onto a selected pixel footprint of the multispectral plasmonic sensor.

Additionally, as shown in FIG. 15, a low numerical aperture objective may maintain near-normal incidence across the pixel area thereby supporting repeatable per-pixel acquisition. In this manner, the optical conditions used to excite the hole-disk plasmonic resonators are controlled for each handedness before spectral measurement is performed.

As shown in FIG. 16, method 300 includes step 306, a chiral active molecule may be introduced onto the surface of the pixelated array of the multispectral plasmonic sensor. In some embodiments, the chiral active molecule (i.e., “analyte”) can be provided as a thin film deposited on a top surface of the pixelated array such that the analyte overlies the one pixels and/or interacts with near-field regions under circularly polarized excitation. Additionally, the analyte may be positioned such that the sensor can be interrogated at multiple pixels without further handling between sequential measurements.

In some embodiments, method 300 comprises step 308, the detection system measures the differential absorption of left-circularly and right-circularly polarized light. In some embodiments, the reflectance spectra of the chiral active molecule are collected for each handedness at a given pixel and/or converted to absorption for dissymmetry computation. Additionally, in some embodiments, the instrument settings, including spectral resolution and/or averaging can be held constant between left- and right-circularly polarized acquisitions at the same pixel to preserve comparability.

As shown in FIG. 16, in some embodiments step 310 of method 300, comprises the detection system that may compute the dissymmetry factors for each pixel of the pixelated array based on the measured spectral responses. In some embodiments, dissymmetry can be computed per pixel from the paired absorption spectra and/or integrated over a spectral interval near the pixel resonance to yield an integrated dissymmetry value.

In some embodiments, method 300 includes step 312, the detection system can assemble the dissymmetry factors into a barcode representing a molecularly unique chiral signature of the introduced chiral active molecule. In some embodiments, the processor operably coupled to the detection system and/or the detection system can determine the integrated dissymmetry of each pixel to the corresponding position in the 2-D grid thereby producing a barcode aligned to the pixel coordinates. Additionally, the barcode and/or associated metadata may be stored for later retrieval and can be compared to a library of stored barcodes for classification or differentiation of enantiomers.

### Method for Fabricating a Multispectral Plasmonic Sensor for Chiral Molecules

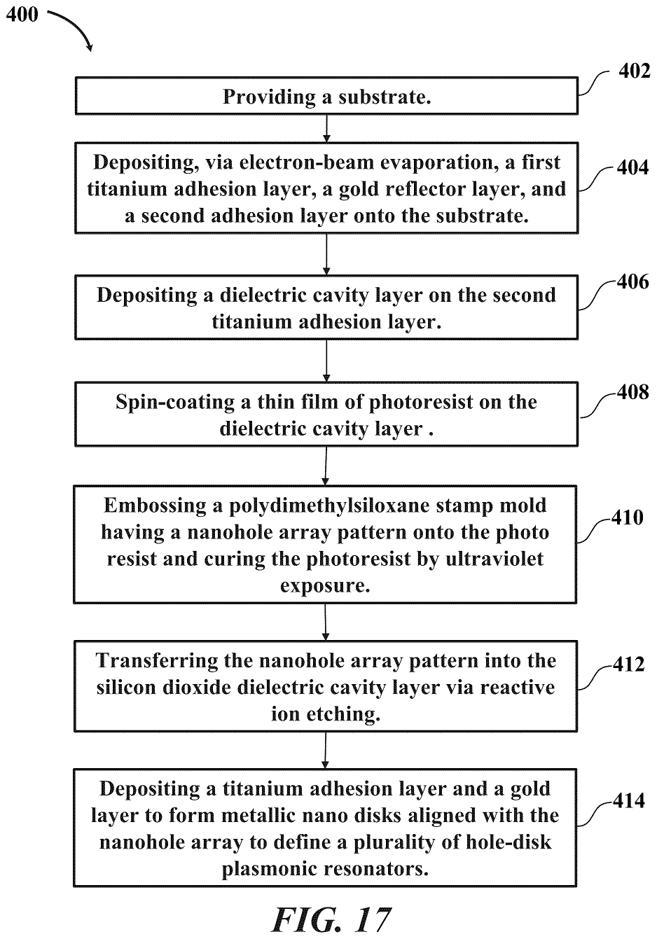

Referring now to FIG. 17, a method 400 is depicted for fabricating a multispectral plasmonic sensor configured for the barcode-based detection described above. The steps delineated in method 400 are merely exemplary of a preferred order of fabrication but may be executed in alternative orders, with additional steps, or with steps omitted depending on the toolset and materials.

As shown in FIG. 17, in conjunction with FIGS. 1-15, method 400 includes step 402, providing a substrate. In some embodiments, the substrate is prepared for deposition utilizing standard deposition methods such as directional deposition and/or pattern transfer compatible with mid-infrared operation of the completed device. Additionally, surface preparation may be performed to support adhesion of the subsequently deposited metallic reflector stack. In this manner, a stable base is established for thin film layering and/or placement and patterning.

In some embodiments, method 400 comprises step 404, a first adhesion layer, a reflector layer, and/or a second adhesion layer are deposited onto the substrate via electron-beam evaporation. In some embodiments, the first and/or second adhesion layer may comprise titanium and/or a titanium alloy. Additionally, the reflector layer may comprise of gold and/or a gold alloy. Accordingly, a mirror-type backplane is formed for reflection-mode operation.

In this manner, the gold layer may perform as a reflector for the mid-infrared band targeted by the pixel resonances. Accordingly, the first and/or second titanium layers may promote adhesion. Additionally, a user may control the thickness of the one or more adhesion layers and/or the reflector layer during deposition to support the optical and/or mechanical characteristics of the reflector layer that underlies the dielectric cavity of the sensor.

Referring to FIG. 17, at step 406, a dielectric cavity layer may be deposited on the second adhesion layer. In some embodiments, the dielectric cavity comprises silicon dioxide that may have a thickness selected to create desired optical phase conditions for resonance with the later-formed pixel structures. Additionally, uniformity of the thickness of the silicon dioxide across the intended pixel field can be maintained to support consistent resonance behavior after pattern formation. In this manner, the dielectric layer may act as a medium into which one or more nanohole arrays may be transferred.

The next step of method 400, step 408, a thin film of photoresist can be spin-coated on the dielectric cavity layer. In some embodiments, spin parameters are selected to obtain a resist thickness compatible with embossing by the poly di-methyl siloxane (“PDMS”) stamp and/or with subsequent reactive ion etching of the dielectric cavity layer.

Proceeding to step 410, a PDMS stamp mold having a nanohole array pattern is embossed into the photoresist film and/or the photoresist film is cured by ultraviolet exposure. In some embodiments, the PDMS stamp encodes the periodic square arrays for all pixels into the sensor as well as the scaling across pixels such that a single embossing step defines the pixelated array in a single process. Additionally, curing may set the photoresist as a protection layer for the dielectric cavity. In this manner, the scaled nanohole geometry is transferred to the photoresist in preparation for etching.

As shown in FIG. 17, method 400 continues with step 412, transferring the nanohole array pattern into the silicon dioxide dielectric cavity layer via reactive ion etching. In some embodiments, etch parameters are selected by a user to achieve a specified and/or desired depth and/or sidewall profile to establish a vertical separation between the metallic nano disks and the underlying apertures. Additionally, in some embodiments, the pixel field is maintained during etching to preserve the scaled geometry encoded by the stamp.

Finally, at step 414, a titanium adhesion layer and/or a gold layer are deposited to form metallic nano disks aligned with the nanohole array of the sensor thereby defining a plurality of hole-disk plasmonic resonators. In some embodiments, alignment between the deposited disks and the etched apertures is provided by the embossed pattern geometry such that the metallic nano disks may overlie the nanoholes of the nanohole array across the pixel grid. Additionally, in some embodiments, deposition of the titanium adhesion layer and/or gold layer is controlled to produce disk features which do not fill the etched apertures, completing the disk-over-hole geometry that supports localized resonances under circularly polarized illumination.

The following examples are provided for the purpose of exemplification and are not intended to be limiting.

### EXAMPLES

### Example #1

### Multispectral Molecular Chiral Barcoding.

### Results

FIG. 1 shows a schematic representation of the pixelated multispectral sensing platform. The sensing surface consists of 25 pixels arranged in a 5×5 array, each having a periodic array of 3D gold-based hole and disk units, stacked on top of an underlying dielectric photonic cavity. The individual pixels cover an area of 1×1 mm² as shown in FIG. 2, with a fixed hole diameter (“D”) and inter-hole periodicity (“P”). The ratio between D and P has been fixed at ⅔ for all pixels to minimize fabrication variability and facilitate ease of scalability. The spectral resonance response of the pixelated system is tuned across a linear range of wavelengths in the mid-IR region. The pixelated sensor shown here is configured to cover a range from 3.3 μm (˜3000 cm⁻¹) to 6.4 μm (1538 cm⁻¹), where most drugs and large biomolecules have their vibrational absorption fingerprints.

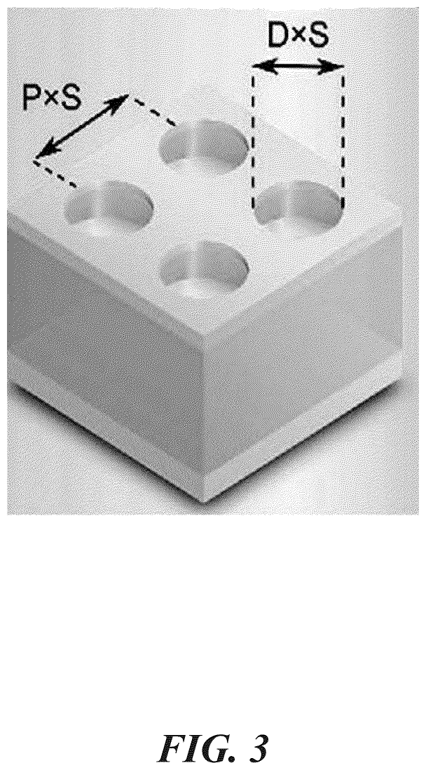

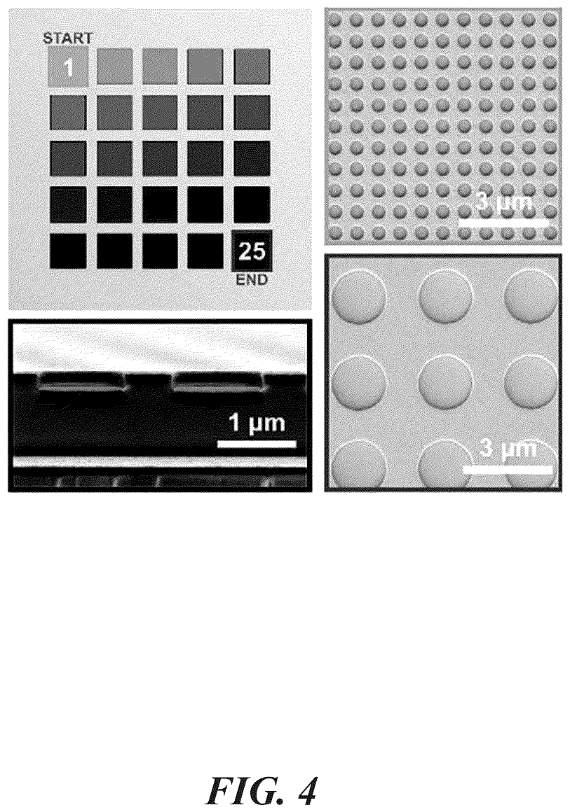

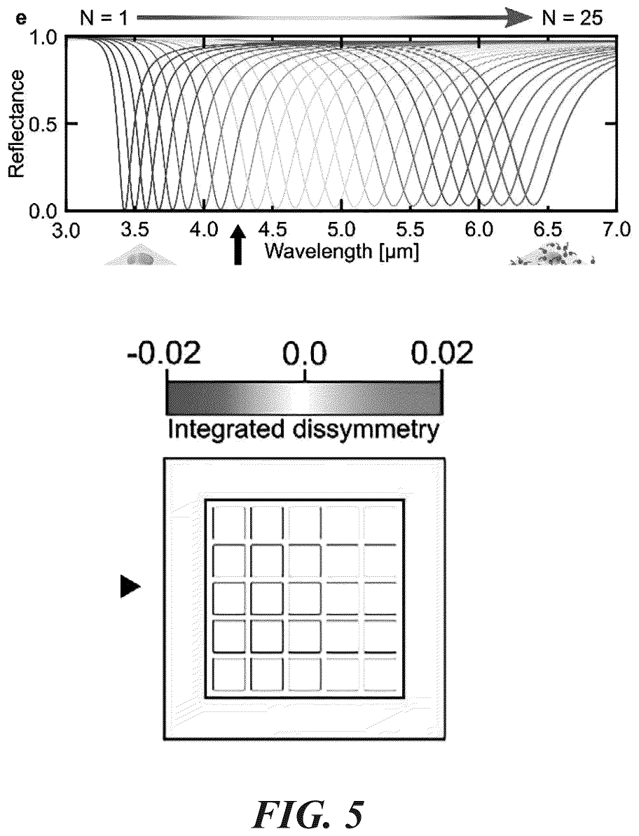

Spectral tunability is controlled by a monotonous scaling parameter S as shown in FIG. 3, with the system's corresponding far-field reflectance spectra shown in FIG. 5. For simplicity, we refer to the pixels by integer values ranging from N=1 to 25, denoting the pixel numbers from top left to bottom right as shown in FIG. 4, top left. FIG. 4 (right top and bottom) shows the top view scanning electron microscope (“SEM”) images for two of the pixels corresponding to the 1st (red square) and 25th (blue square). The plasmonic array is coupled to an underlying resonant cavity formed by a reflector at the bottom, as seen in the cross-sectional SEM image in FIG. 4 (left bottom). The hybrid coupling between the top localized surface plasmon resonance (“LSPR”) mode of the 3D hole-disk array and the photonic cavity resonant mode generates a sharp and narrow hybridized resonance characterized by a near-perfect absorption (dip in reflectance) in the far-field spectral domain.

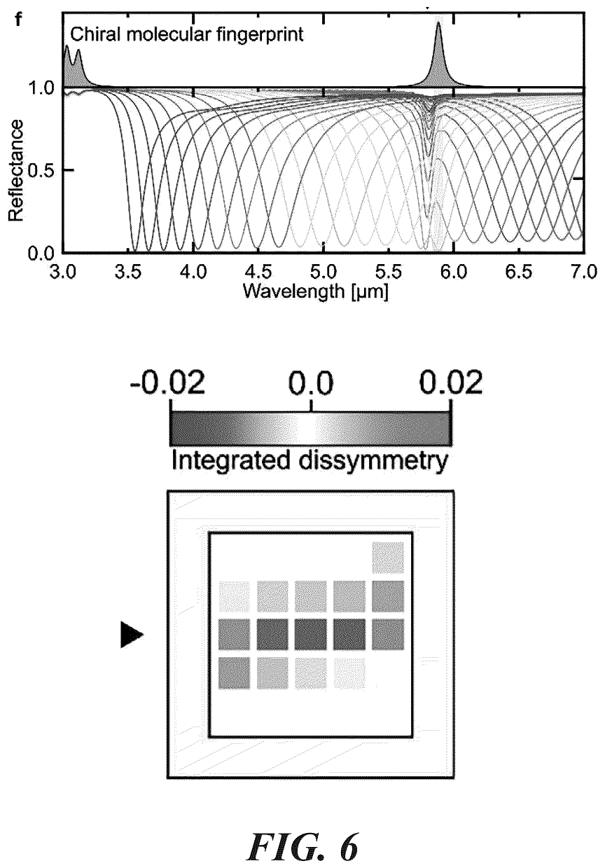

The system's geometrical parameters are optimized in a way to have the lowest (first) order resonance response in the mid-IR region, and the subsequent pixel array responses are scaled accordingly. FIG. 5 shows the far-field reflectance spectra in the mid-IR for the bare nanostructured pixels. The corresponding reflectance response after adsorption of a chiral active molecule on the pixels is shown in FIG. 6, along with its inherent IR absorption spectra. The corresponding calculated chiral barcodes based on the integrated dissymmetry are shown in FIGS. 5-6, respectively, where the dissymmetry factor (g) is denoted as g=2(A⁺−A⁻)/(A⁺+A⁻) corresponding to the absorption (“A”) for right (+) and left (−) CPL. While the generated barcode corresponding to a bare sensor shows null response, as shown in FIG. 5, the one with the adsorbed chiral molecule shows a distinct response with highlighted pixels, characteristic of the molecule's chiral behavior.

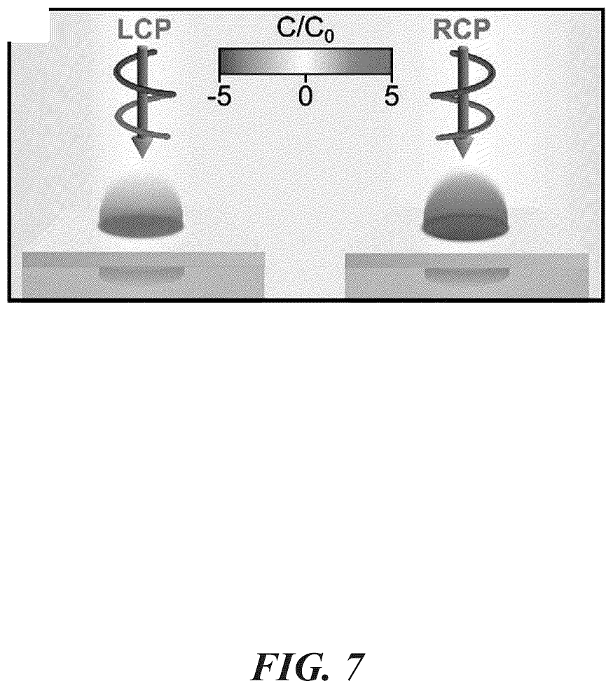

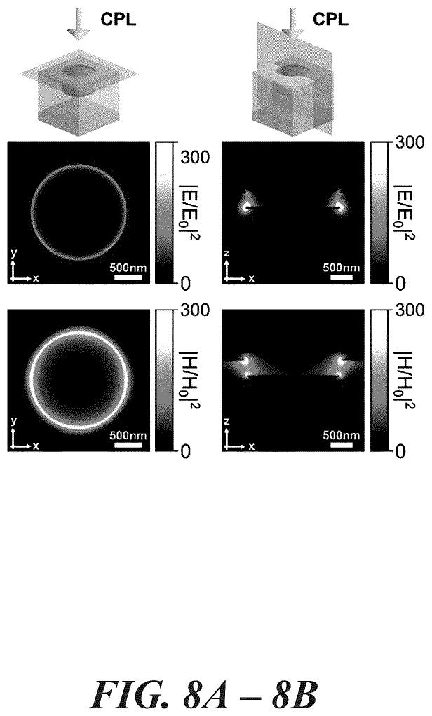

On excitation with CPL, simultaneous electric and magnetic modes are generated in the near-field at resonance/These resonant modes are analogous to a rotating dipole with a spatially symmetric magnitude over the patterned surface. The polarity of this near-field is flipped based on the handedness of the excitation CPL as can be observed from the finite difference time domain (FDTD) predictions in FIG. 7. FIGS. 8A-8B show the simulated time-averaged electric and magnetic field intensity enhancements respectively at resonance for one of the pixelated arrays under CPL excitation. The magnetic mode enhancement is stronger near the hole region, while the electric field strength peaks near the disk region within the geometry. The field intensity distribution is identical for both handedness of the circularly polarized excitation.

This unique spatial distribution generates an enhanced chiral density (“C”), described as a conserved pseudoscalar that quantifies the magnitude of “helical twist” of light. C is defined for a monochromatic light source as

$$C ( ω ) = - \frac{{ε}_{0} ω}{2} Im ( {E}^{*} \cdot B ) ,$$

where E* is the complex conjugate of the local electric field, B is the corresponding magnetic field, ω is the frequency and ϵ₀ is the permittivity of free space. FIG. 7 shows the distribution of enhanced optical chirality over the plasmonic system under left (“LCP”) and right (“RCP”) handed circularly polarized illumination. The optical chirality enhancement (“Cnf”) is defined as C normalized to the free-space optical chiral density (“CCPL”). This exhibits two important qualities of the system, a) the optical chirality enhancement has a homogenous magnitude that solely depends on the rotational handedness of the excitation, and b) the enhancement is purely a near-field effect and does not impart a far-field CD or ORD response to the system.

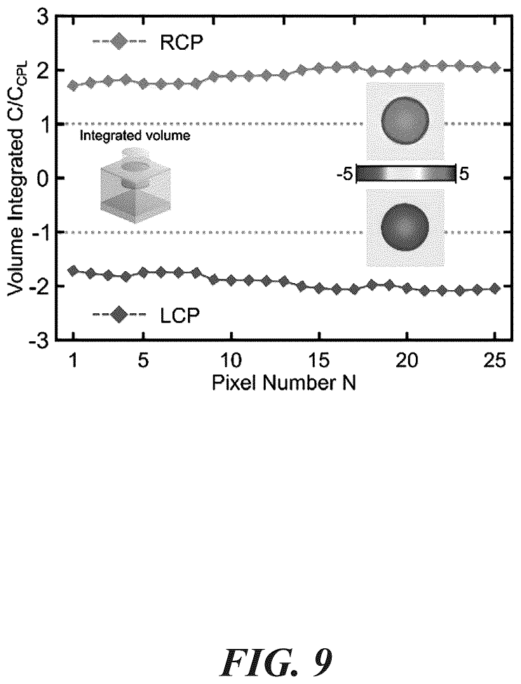

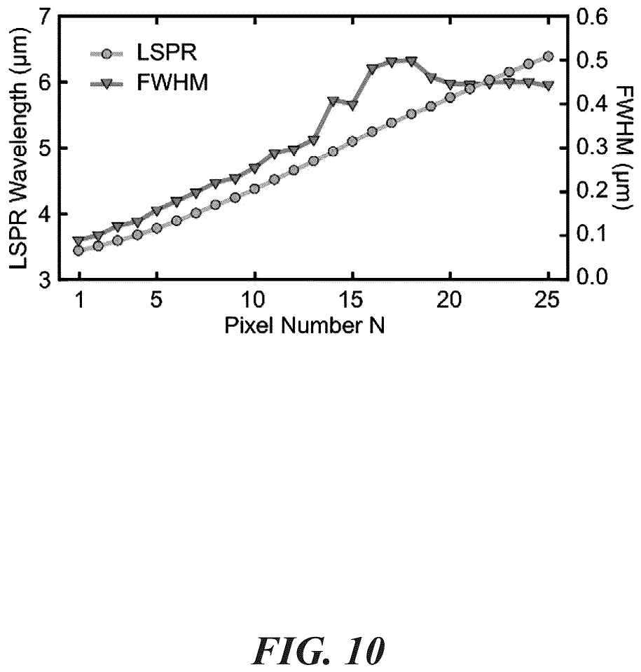

This allows suppression of the structure-induced asymmetry and ensures that any asymmetric response on molecular adsorption is only due to the chiral properties of the adsorbed analytes. Moreover, the resonance tuning capability allows simultaneous spectral tuning of the near-field electric and chiral density modes. FIG. 9 shows the volume integrated optical chirality enhancement at resonances as a function of the pixel number, for which the details of calculation are provided in the supplementary information. The corresponding resonance wavelengths and full width half maxima (“FWHM”) are shown in FIG. 10. The vertical distance of integration is taken up to 200 nm above the top metallic layer, as this encompasses typical thickness ranges of the adsorbed molecular layers. The inset in FIG. 9 shows a representative 2D-distribution of Cnf for one of the pixels, 2 nm above the top surface layer for RCP (top) and LCP (bottom) illumination. The near-field exhibits high chiral enhancement (Cnf>1) over the sensing layer, indicating an enhanced chiral density magnitude at the first order resonances for all pixelated arrays.

Since the proposed sensing platform has a bottom mirror, the incident light is either absorbed by the top plasmonic system or reflected back to free space. As a result, the absorption spectrum can simply be calculated as A=1−R, where A and R denote the absorption and reflection coefficients. Typical spectral measurements are performed over a macroscopically significant area over which the chiral molecules are adsorbed. As such, the adsorbed molecules can be considered as a randomly oriented isotropic ensemble. The absorption rate for chiral molecules of such an isotropic system in the presence of monochromatic CPL having frequency ω is given as:

$${A}^{( + / - )} = \frac{ω}{2} ( {α}^{″} {\vert \tilde{E} \vert}^{2} + {χ}^{″} {\vert \tilde{B} \vert}^{2} ) \pm {G}^{″} ωIm ( {\tilde{E}}^{*} \cdot \tilde{B} ) \qquad ( 1 )$$

Here, superscripts + and − indicate RCP and LCP excitation, α″ and χ″ are the imaginary parts of the electric polarizability and magnetic susceptibility of the chiral molecule respectively, while G″ denotes the mixed electric-magnetic dipolar response which is non-zero for chiral active molecules, and \tilde{E} and \tilde{B} are the complex local electric and magnetic fields at the molecule respectively. For a general non-magnetic system (valid for most naturally occurring molecules), the expression for dissymmetry factor (g) becomes:

$$g = ( \frac{{G}^{″}}{{α}^{″}} ) ( \frac{2 C}{{ωε}_{0} {\vert E \vert}^{2}} ) \qquad ( 2 )$$

The contribution from the terms involving magnetic susceptibility is ignored as most molecules are non-magnetic in nature. The dissymmetry factor depends on both the optical property of the molecules (first term in parenthesis) and the electromagnetic properties of the surrounding field (second term in parenthesis). The dimensional mismatch between molecules and excitation wavelengths (few nm vs 100's of nm) renders the first term very small. Therefore, by proper engineering of the nanostructured system, the field-dependent term can be improved by enhancing C. This constitutes the main advantage of our proposed plasmonic platform, providing simultaneous enhancements in near-field \tilde{E} and C. Additionally, the \tilde{E} enhancement is more localized near the bottom disk region than the top hole, which consequently would lead to an overall enhancement in the dissymmetry factor at resonance for the coupled molecule-plasmonic system (E being in the denominator).

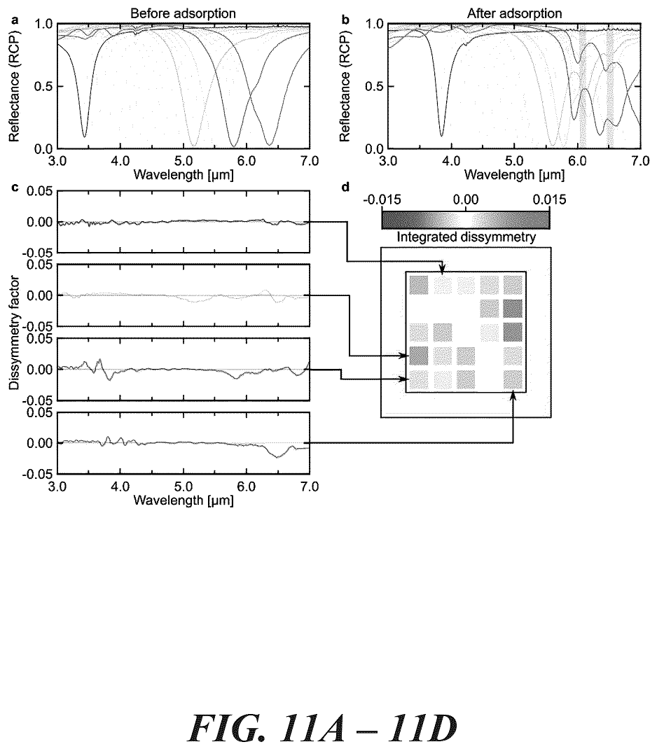

On molecular adsorption, an asymmetric CD response is generated when there is a significant overlap between the molecular absorption and the plasmonic resonance. FIGS. 11A-11D show the experimentally measured far-field spectral reflectance for the pixelated array sensor before and after molecular layer adsorption respectively (shown here just for RCP excitation). Here, biomolecule lysozyme was used as an example to demonstrate chiral barcode formation. Lysozyme has two prominent absorption bands corresponding to Amide I (1658 cm⁻¹) and Amide II (1530 cm⁻¹), highlighted in FIG. 11B as two gray bars for better visualization. Note that for the bare sensor in FIG. 11A, there is a slight resonance broadening at longer wavelengths (e.g., red, and purple curves in FIG. 11A) due to the inhomogeneous linewidth broadening of the LSPR mode.

Nevertheless, both LCP and RCP excitations produce identical resonances resulting in zero far-field CD. On molecular adsorption, the far-field reflectance R changes as shown in FIG. 11B which eventually indicates a change in absorption A between LCP and RCP excitation. The difference in LCP and RCP absorption is represented in the dissymmetry factor g. Primarily, the dissymmetry factor enhances intensity differences by normalizing with respect to the detected signal, providing a quantitative analysis of the degree of differential absorption.

Four cases of various degrees of overlaps between the molecular absorption and sensor resonances are highlighted as blue, green, red, and magenta color plots in FIGS. 11A-11B. The molecular adsorption is accompanied by a spectral shift that depends on the local changes in refractive index and molecular density on the sensor surface, a common effect that occurs due to the reduction in the light's phase velocity in effective near-field media, lowering the wavevector and leading to change of resonance condition to lower energies, effectively seen as a spectral red-shift.

The spectral splitting, however, occurs due to the coherent interference between two resonant absorption modes that generates a plasmon-induced transparency (“PIT”) at the overlap location. This occurs due to a degeneracy-induced interference between a sub-radiant (dark) and a super-radiant (bright) mode, which in this case corresponds to the weak molecular absorption and the sensor's strong plasmon resonance, respectively. The strength of the induced splitting is dictated by the molecular density and the degree of spectral overlap between the molecule and the corresponding pixels' resonant absorptions.

On excitation with oppositely-handed CPL, different spectral dissymmetry factors are observed for all the pixels. These variations in spectral overlap would lead to different degrees of coupling between the near-field chiral enhancement and the inherent molecular chirality, leading to variations in the measured VCD far-field response. FIG. 11C shows the baseline subtracted dissymmetry factors for four pixels corresponding to the four color-coded spectra in FIG. 11B. A weaker overlap would produce a relatively weak SEVCD response as seen in the dissymmetry spectra as depicted in FIG. 11C. Additionally, near-field chiral coupling determines the magnitude as well as the sign of the observed dissymmetry response (+g/−g).

As mentioned before, due to the geometrical symmetry of the system, the dissymmetry response should be zero in absence of chiral molecules for all pixels. However, due to slight imperfections in fabrication of the sensors, experimentally determined dissymmetry spectra exhibit slight non-zero values near resonances even without a molecular layer. This is taken as a baseline (g_{w/o. molecule}) and is subtracted from the calculated dissymmetry from the molecule-coated sensor's responses (g_{w. molecules}) to get a final background removed dissymmetry spectra (g=g_{w. molecules}−g_{w/o. molecule}), containing only chiral information from the molecule. Moreover, a quantity gᵢₙₜ, defined as gᵢₙₜ=∫gdλ, was specified, where the integration is over a spectral region encompassing the molecule-induced perturbation in the sensor's spectral response. Non-overlapping or weakly overlapping pixels would produce negligible gᵢₙₜ. For subsequent pixels, the dissymmetry spectra would be modified accordingly, leading to different magnitudes and signs for gᵢₙₜ, depending on the near-field chiral interaction. The associated gᵢₙₜ values are color-coded and represented as a “chiral barcode,” that is uniquely assigned to a chiral molecule as shown in FIG. 11D. The barcode pattern depends on the degree of molecule-sensor pixel spectral absorption overlap. If M color codes can be discerned and there are N pixels, then M!*N! molecules can be uniquely identified based on N sensor pixels.

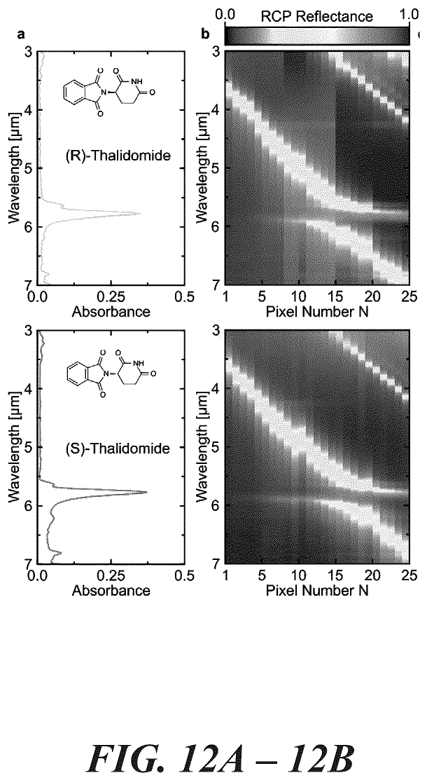

The first approach shown is to utilize this barcoding platform to perform enantio-selectivity, i.e., to identify enantiomers. For this study, thalidomide enantiomers are chosen as target molecules for classification. FIG. 12A shows the spectral absorbance of (R)-thalidomide (top) and (S)-thalidomide (bottom) adsorbed on optically thick gold-coated substrates in the region of interest, measured using infrared reflection-absorption spectroscopy (“IRRAS”). A thin layer of thalidomide molecules is adsorbed on the surface of the multispectral sensor array, the adsorption process is described in the Methods Section.

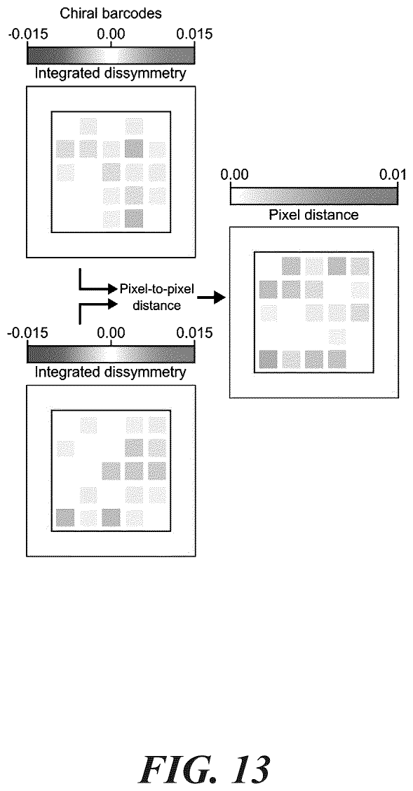

The determined thickness, verified with an average topology measurement of the adsorbed sensor surface using atomic force microscopy, is ≈160 nm. The corresponding SEIRA-based reflectance for the RCP excitation, for both (R) and (S)-thalidomide, is shown as a 2D heatmap in FIG. 12B. A PIT-induced splitting can be observed at the region of sharp molecular absorption (≈5.79 μm, corresponding to the C═O stretch vibration in thalidomide) indicating strong near-field coupling between the molecular absorption response and the underlying sensor plasmon resonance. The corresponding generated barcodes for the two enantiomeric molecules are shown in FIG. 13. The barcode for (R)-thalidomide, as shown in FIG. 13, shows mostly red pixels, indicating that the “weighted” dissymmetry is mostly positive.

Similarly, the barcode for (S)-thalidomide, as expected, exhibits an opposite response, as seen in FIG. 13 (bottom). For a fair comparison, the same sensor has been used for both measurements after thorough cleaning (Methods Section) and verification of cleaned spectral response. The pixel-to-pixel distance is represented as a differential barcode in FIG. 13, indicating the difference in the integrated dissymmetry value for each pixel. A brighter color, corresponding to a larger pixel-to-pixel distance, indicates greater chiral response dissymmetry at that spectral location.

In the next study, it was demonstrated the identification of larger biomolecules that vary in their physical and biological properties but have similar vibrational absorption finger-prints. Such biomolecules cannot be identified by simple SEIRA-based techniques but can be distinguished based on their chiral response. Hemoglobin and Lysozyme were chosen as tar-get proteins for this study. Hemoglobin, a protein found in red blood cells, facilitates transportation of oxygen in living beings, while lysozyme is another lighter protein that helps in providing immunity against external microorganisms.

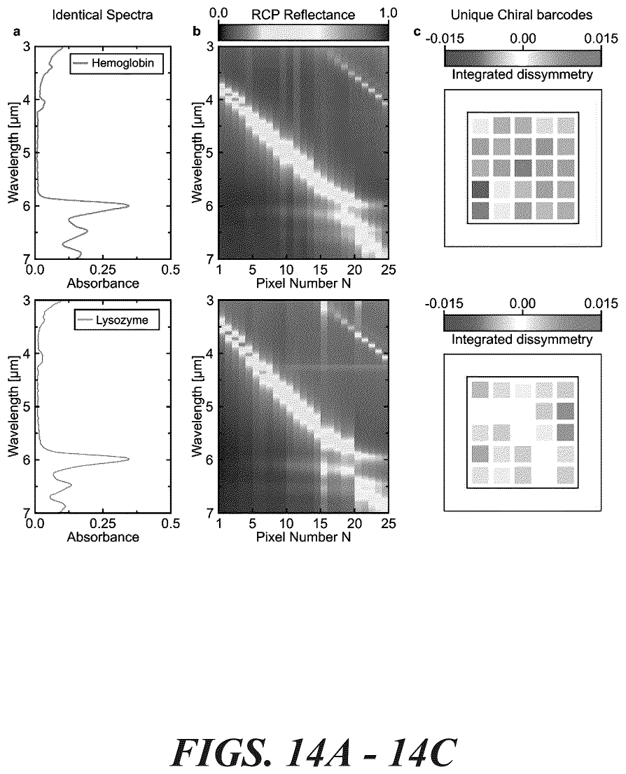

Hemoglobin (≈64 500 g mol⁻¹) is ≈4.5 times heavier than lysozyme (≈14 000 g mol⁻¹) while having a similar absorption profile in the mid-infrared region. FIG. 14A shows the measured IRRAS for both biomolecules, hemoglobin (top) and lysozyme (bottom) adsorbed on gold substrates. Both have similar absorption spectra in the mid-infrared domain, with absorption bands at 1640 and 1580 cm⁻¹. The corresponding SEIRA-based 2D heatmaps for RCP excitation on molecular adsorption are shown in FIG. 14B, exhibiting a similar trend in terms of the PIT-based splitting observed at the locations of resonant overlaps.

The subtle reflectance differences observed in pixels 15 to 25 are due to slight differences in film thicknesses, experimental conditions, and sensor used during the measurements. However, the spectral splitting at the position of molecular and LSPR overlap for both cases occur at the same locations due to both biomolecules having similar absorption fingerprint, hence it can-not be used to distinguish them uniquely. While the concentration for both biomolecular solutions are same, the absorbed hemoglobin layer corresponds to a thickness of ≈120 nm, while for the lysozyme, the average surface thickness is measured to be ≈100 nm, as determined by AFM-based averaged surface topology measurements.

The appropriate baseline corrected integrated dissymmetry-based barcodes are shown in FIG. 14C corresponding to hemoglobin (top) and lysozyme (bottom). Interestingly, the barcodes show distinct values for both biomolecules, which uniquely identify them depending on their chiral interaction with spectrally varying pixels. Hemoglobin shows integrated dissymmetry with mostly positive values as indicated by the reddish pixels. On the other hand, the trend for lysozyme seems to be more balanced with higher wavelength pixels showing a blueish color, indicating a mostly negative value of integrated dissymmetry.

As the pixel resonances, and in turn the enhanced chiral fields, are swept over the molecular fingerprint region, different degrees of overlap occur between the molecular chiral response and the plasmon-enhanced chiral near-field, leading to differences in the chiral light-matter interactions, which are reflected in the (sign and magnitude) integrated dissymmetry values. Note that for some pixels with plasmon resonances at the higher wavelengths, certain higher-order resonances appear in the range of interest, as shown in FIG. 13B and FIG. 14B which also generate enhanced chiral near-field due to simultaneous higher order modes interaction. But these resonances are non-overlapping with any of the vibrational fingerprint regions in question and hence would con-tribute negligibly to the overall dissymmetry.

### CONCLUSION

The ability to utilize the concept of optical chirality for sensitive detection and classification of chiral active molecules opens a new paradigm for molecular identification. Here, a plasmonic multispectral pixelated array exhibiting hybrid localized plasmon resonance in the mid-IR wavelength range to create unique chiral-based molecular barcodes based on their absorption finger-prints has been proposed and experimentally demonstrated. The nanostructured platform generates simultaneously enhanced localized and symmetric electric and magnetic near-field mode at resonance on excitation with CPL. This in turn generates enhanced near-field optical chiral density which is homogeneous and whose sign can be controlled by the polarization handedness of the excitation without requiring multiple separate chiral nanostructures. The system's spectral resonance, and in turn its enhanced chiral response, can be tuned by changing the system's geometrical parameters, which has been utilized here to generate a multispectral array-based sensor.

SEVCD, or VCD in general, provides valuable information about molecular structure and conformations that would not be possible in the UV-visible region. Owing to the system's symmetry and local nature of the enhancements, SEIRA and SEVCD measurements simultaneously were demonstrated using this platform, enabling not only extract information about the molecules IR chemical properties, but also provide structural information that facilitates enantiomeric discrimination and higher-order structure identification based on a unique chiral barcoding scheme.

To demonstrate the efficacy of the scheme, the platform was experimentally distinguished between an enantiomeric pair of small molecules and two spectroscopically similar large biomolecules based on their unique chiral barcodes. The fabrication process was simple and amenable to batch production using common techniques like nanoimprint lithography and standard deposition methods. Furthermore, the response of the nanostructured system can be readily extended through parametric scaling to encompass a broader range, from mid to far infrared regions, aligning with the absorption fingerprint of the targeted chiral drugs or biomolecules. The enhanced resonant chirality combined with geometrical spectral tunability of the nanostructured pattern allows us to span a large region for reliable molecular barcoding. It is envisioned that this platform can be integrated to dynamically monitor real-time changes in protein dynamics and create unique libraries of chiral barcodes based on their IR fingerprints. Such a versatile system can be a useful tool for identification and classification of biomolecules and synthetic drugs, having relevance in chemical, medical, and pharmaceutical research.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. A multispectral plasmonic sensor for detecting and characterizing chiral-active molecules, comprising:
a substrate;
a reflector layer disposed about a top surface of the substrate;
a dielectric cavity layer disposed about a surface of the reflector layer;
a pixelated array comprising a plurality of pixels disposed about a surface of the dielectric cavity layer wherein each pixel of the pixelated array comprises a periodic square array of nanoholes;
a plurality of metallic nano disks disposed above the nanoholes of the pixels of the pixelated array to define a plurality of hole-disk plasmonic resonators; and
wherein the pixels of the pixelated array are scaled in increments across the array such that each pixel can resonate at a distinct mid-infrared (“mid-IR”) wavelength.

2. The sensor of claim 1, wherein the pixelated array comprises a 5 by 5 array of pixels.

3. The sensor of claim 1, wherein the nanoholes of each pixel of the pixelated array comprises a dimensional size of 1×1 mm.

4. The sensor of claim 1, wherein the metallic reflector layer comprises a first titanium adhesion layer, a gold layer reflector layer, and a second titanium adhesion layer.

5. The sensor of claim 1, wherein the dielectric cavity layer comprises silicon dioxide.

6. The sensor of claim 1, wherein the pixels are scaled by a predetermined amount by incrementally increasing the nanohole size of the pixels and periodicity by 5% between each successive pixel.

7. The sensor of claim 1, wherein the nanoholes of the pixels have a fixed ratio of diameter to periodicity of 2:3 across the pixelated array.

8. A system for generating a molecular barcode of a chiral active molecule, comprising:
a multispectral plasmonic sensor comprising:
a substrate;
a reflector layer disposed about a top surface of the substrate;
a dielectric cavity layer disposed about a surface of the reflector layer;
a pixelated array comprising a plurality of pixels disposed about a surface of the dielectric cavity layer wherein each pixel of the pixelated array comprises a periodic square array of nanoholes;
a plurality of metallic nano disks disposed above the nanoholes of the pixels of the pixelated array to define hole-disk plasmonic resonators; and
wherein the pixels of the pixelated array are scaled in increments across the array such that each pixel can resonate at a distinct mid-infrared (“mid-IR”) wavelength;
a light source configured to generate mid-IR circularly polarized light directed towards the pixelated array; and
a detection system communicatively coupled to the pixelated array configured to measure spectral responses of the pixelated array under left-circularly polarized and right-circularly polarized illuminations and compute dissymmetry factors for the pixelated array and assemble the computed dissymmetry factors into a chiral barcode.

9. The system of claim 8, wherein the detection system comprises:
a reflection-mode microscope having a low numerical aperture;
a Fourier transform infrared spectrometer operably coupled to the reflection-mode microscope having a mid-IR thermal source;
a quarter-wave plate mechanically coupled to a motorized rotation stage configured to generate left-handed and right-handed circularly polarized excitation; and
a cryo-cooled mercury-cadmium telluride detector configured to collect reflected responses.

10. The system of claim 8, wherein the Fourier transform infrared spectrometer comprises a potassium bromide beamsplitter.

11. The system of claim 8, further comprising a processor configured to compare the chiral barcode of the chiral active molecule to a library of stored chiral barcodes to classify the chiral active molecule.

12. The system of claim 8, wherein the barcode represents integrated dissymmetry values across the plurality of pixels.

13. The system of claim 8, wherein the light source comprises a broadband mid-IR thermal source.

14. A method for detecting and characterizing chiral active molecules, the method comprising:
providing a system for generating a molecular barcode of a chiral active molecule, the system comprising:
a multispectral plasmonic sensor;
a light source configured to generate mid-IR circularly polarized light directed towards the pixelated array; and
a detection system communicatively coupled to the pixelated array configured to measure spectral responses of the pixelated array under left-circularly polarized and right-circularly polarized illuminations and compute dissymmetry factors for the pixelated array and assemble the computed dissymmetry factors into a chiral barcode;
illuminating the multispectral plasmonic sensor with circularly polarized light to generate enhanced electric and magnetic near fields at each pixel of the pixelated array of the plasmonic sensor;
introducing a chiral active molecule onto the surface of the pixelated array of the multispectral plasmonic sensor;
measuring, via the detection system, the differential absorption of left-circularity and right-circularly polarized light;
computing, via the detection system, dissymmetry factors for each pixel of the pixelated array based on the measured spectral responses; and
assembling, via the detection system, the dissymmetry factors into a barcode representing a molecularly unique chiral signature of the introduced chiral active molecule.

15. The method of claim 14, wherein the detection system comprises:
a reflection-mode microscope having a low numerical aperture;
a Fourier transform infrared spectrometer operably coupled to the reflection-mode microscope having a mid-IR thermal source;
a quarter-wave plate mechanically coupled to a motorized rotation stage configured to generate left-handed and right-handed circularly polarized excitation; and
a cryo-cooled mercury-cadmium telluride detector configured to collect reflected responses.

16. The method of claim 14, wherein the chiral active molecule is provided as a thin film deposited on a top surface of the pixelated array.

17. The method of claim 14, wherein assembling the dissymmetry factors into a barcode comprises integrating dissymmetry factors across a spectral region corresponding to molecular absorption bands.

18. The method of claim 14, wherein the light source comprises a broadband mid-IR thermal source.

19. The method of claim 14, further comprising the step of differentiating, via the detection system and chiral barcode, enantiomers of the chiral molecule based on distinct spectral profiles.

20. The method of claim 14, further comprising the step of calibrating the multispectral plasmonic sensor prior to introducing the chiral active molecule by measuring baseline spectral responses of the pixelated array under left- and right-circularly polarized illumination without any analyte.
